(19) United States
(12) Patent Application Publication
(10) Pub. No.: US 2018/0303512 A1
(43) Pub. Date: Oct. 25, 2018

(54) DEVICES, SYSTEMS, AND METHODS FOR DELIVERING A THERAPY TO A TARGET TISSUE

(71) Applicant: OHIO STATE INNOVATION FOUNDATION, Columbus, OH (US)

(72) Inventors: Ali REZAI, Morgantown, WV (US); Milind DEOGAONKAR, Westerville, OH (US)

(21) Appl. No.: 15/779,318

(22) PCT Filed: Nov. 25, 2016
(86) PCT No.: PCT/US2016/063756

§ 371 Date: May 25, 2018

Related U.S. Application Data
(60) Provisional application No. 62/260,012, filed on Nov. 25, 2015.

(51) Int. Cl. A61B 17/34; A61B 1/05

(52) U.S. Cl. CPC A61B 17/3401; A61B 17/3468; A61B 1/05; A61B 17/3403

(57) ABSTRACT

A method for guiding a therapy delivery device to a target tissue in a subject can include a step of providing a needle (12) having a main body that defines an inner lumen. An opening (26) can be located on a distal end portion of the main body. The opening can be in communication with the inner lumen. A ramp (32) can be positioned within the inner lumen and adjacent the opening. The ramp can be axially spaced apart from a distal end portion of the main body. The needle can be percutaneously inserted into the subject. The needle can be advanced to the target tissue. A camera (68) can be advanced through the inner lumen. The camera can be operated to visualize the target tissue. A therapy delivery device (70) can be advanced through the inner lumen. The therapy delivery device, under direct visualization, can be placed into communication with the target tissue.

### RELATED APPLICATION

This application claims the benefit of U.S. Provisional Patent Application Ser. No. 62/260,012, filed Nov. 25, 2015, the entirety of which is hereby incorporated by reference for all purposes.

### TECHNICAL FIELD

The present disclosure relates generally to devices, systems, and methods for percutaneous surgical or medical procedures and, more particularly, to devices, systems, and methods for percutaneously delivering a therapy to a target tissue in a subject.

### BACKGROUND

At present, an epidural procedure is done using a standard Touhey epidural needle under fluoroscopic guidance. Guidance of the epidural needle is thus based on fluoroscopy and tactile feedback. This presents several drawbacks, however, including extended radiation/fluoroscopy exposure, increased risk of dura layer puncture and overall longer procedure time, all of which raise the level of patient risk associated with such procedures.

### SUMMARY

The present disclosure relates generally to devices, systems, and methods for percutaneous surgical or medical procedures and, more particularly, to devices, systems, and methods for percutaneously delivering a therapy to a target tissue in a subject.

One aspect of the present disclosure can include a percutaneous needle for guiding a therapy delivery device to a target tissue in a subject. The needle can comprise a main body defining an inner lumen, an opening, and a ramp. The opening can be located on a distal end portion of the main body and be in communication with the inner lumen. The ramp can be positioned within the inner lumen and adjacent the opening. The ramp can be axially spaced apart from a distal end of the main body.

Another aspect of the present disclosure can include an epidural needle system. The epidural needle system can consist of a percutaneous needle and a camera. The percutaneous needle can include a main body defining an inner lumen and an opening located on a distal end portion of the main body. The opening can be in communication with the inner lumen. The needle can also include a ramp positioned within the inner lumen and adjacent the opening. The ramp can be axially spaced apart from a tip of the main body. The camera can be slidably disposed within the inner lumen. The camera can have a distal end portion that overlies, and is in direct contact with, the ramp such that the ramp deflects the camera radially away from the distal end portion of the needle.

Another aspect of the present disclosure relates to a method for guiding a therapy delivery device to a target tissue in a subject. One step of the method can include providing a needle having a main body that defines an inner lumen. An opening can be located on a distal end portion of the main body. The opening can be in communication with the inner lumen. A ramp can be positioned within the inner lumen and adjacent the opening. The ramp can be axially spaced apart from a distal end portion of the main body. The needle can be percutaneously inserted into the subject. The needle can be advanced to the target tissue. A camera can be advanced through the inner lumen. The camera can be operated to visualize the target tissue. A therapy delivery device can be advanced through the inner lumen. The therapy delivery device, under direct visualization, can be placed into communication with the target tissue.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a perspective view of a percutaneous needle constructed in accordance with one aspect of the present disclosure;

FIG. 2 is a cross-sectional view taken along Line 2-2 in FIG. 1;

FIG. 3 is a perspective view of a percutaneous needle constructed in accordance with another aspect of the present disclosure;

FIG. 4 is a cross-sectional view taken along Line 4-4 in FIG. 3;

FIG. 5 is a process flow diagram illustrating a method for applying a therapy to a target tissue according to another aspect of the present disclosure;

FIG. 6 is a schematic illustration showing the percutaneous needle in FIG. 1 being inserted into a target tissue;

FIG. 7 is a perspective view showing a first surgical instrument extending through an opening of the percutaneous needle in FIG. 6;

FIG. 8 is a cross-sectional view taken along Line 8-8 in FIG. 7;

FIG. 9 is a schematic illustration showing a first surgical instrument extending through an opening of the percutaneous needle in FIG. 8;

FIG. 10 is a perspective view showing a second surgical instrument extending through an opening of the percutaneous needle in FIG. 9;

FIG. 11 is a cross-sectional view taken along Line 11-11 in FIG. 10; and

FIG. 12 is a schematic illustration showing a second surgical instrument extending through an opening of the percutaneous needle in FIG. 11.

### DETAILED DESCRIPTION

Definitions

Unless defined otherwise, all technical and scientific terms used herein have the same meaning as is commonly understood by one of skill in the art to which the present disclosure pertains.

In the context of the present disclosure, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” and “between about X and Y” can be interpreted to include X and Y.

As used herein, phrases such as “between about X and Y” can mean “between about X and about Y.”

As used herein, phrases such as “from about X to Y” can mean “from about X to about Y.”

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms can encompass different orientations of the device in use or operation in addition to the orientation depicted in the figures. For example, if the device in the figures is inverted, elements described as “under” or “beneath” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

As used herein, the term “subject” can be used interchangeably with the term “patient” and refer to any warm-blooded organism including, but not limited to, human beings, pigs, rats, mice, dogs, goats, sheep, horses, monkeys, apes, farm animals, livestock, rabbits, cattle, etc.

As used herein, the term “target tissue” can refer to a desired portion of biological tissue (e.g., spinal nervous tissue) to which a therapy can be applied.

As used herein, the phrase “spinal nervous tissue” can refer to nerves, neurons, neuroglial cells, glial cells, neuronal accessory cells, nerve roots, nerve fibers, nerve rootlets, parts of nerves, nerve bundles, mixed nerves, sensory fibers, motor fibers, dorsal root, ventral root, dorsal root ganglion, spinal ganglion, ventral motor root, general somatic afferent fibers, general visceral afferent fibers, general somatic efferent fibers, general visceral efferent fibers, grey matter, white matter, the dorsal column, the lateral column, and/or the ventral column associated with the spinal cord.

As used herein, the term “therapy” can refer to a substance, material, or device for therapeutically regulating, preventing, improving, alleviating the symptoms of, reversing and/or reducing the effects of an undesired characteristic of a target tissue.

As used herein, the terms “modulate” or “modulating” with reference to a target tissue (e.g., spinal nervous tissue) can refer to causing a change in neuronal activity, chemistry, and/or metabolism. The change can refer to an increase, decrease, or even a change in a pattern of neuronal activity. The terms may refer to either excitatory or inhibitory stimulation, or a combination thereof, and may be at least electrical, magnetic, optical or chemical, or a combination of two or more of these. The terms “modulate” or “modulating” can also be used to refer to a masking, altering, overriding, or restoring of target tissue activity.

As used herein, the term “in communication” can refer to at least a portion of a component, element, or structure being adjacent, in the general vicinity, in close proximity, and/or directly next to a second component, element, or structure.

As used herein, the term “electrical communication” can refer to the ability of an electric field generated by an electrode or electrode array to be transferred, or to have a neuromodulatory effect, within and/or on at least one nerve, neuron, and/or nervous tissue of a target tissue (e.g., spinal nervous tissue.)

### Overview

The present disclosure relates generally to devices, systems, and methods for percutaneous surgical or medical procedures and, more particularly, to devices, systems, and methods for percutaneously delivering a therapy to a target tissue in a subject. Conventional epidural needles are guided to their targets using fluoroscopy and tactile feedback. Epidural needles, however, do not include a guidance mechanism for visualizing the needle within the epidural space to avoid puncture of the dura layer. Advantageously, the present disclosure provides a percutaneous needle that: (1) reduces the use of extended fluoroscopy; (2) permits visualization of spinal nervous tissue, such as the yellow ligament and the dura layer; (3) reduces the risk of dura layer puncture; and (4) is capable of accommodating at least one surgical instrument for application of a therapy to a target tissue.

### Devices

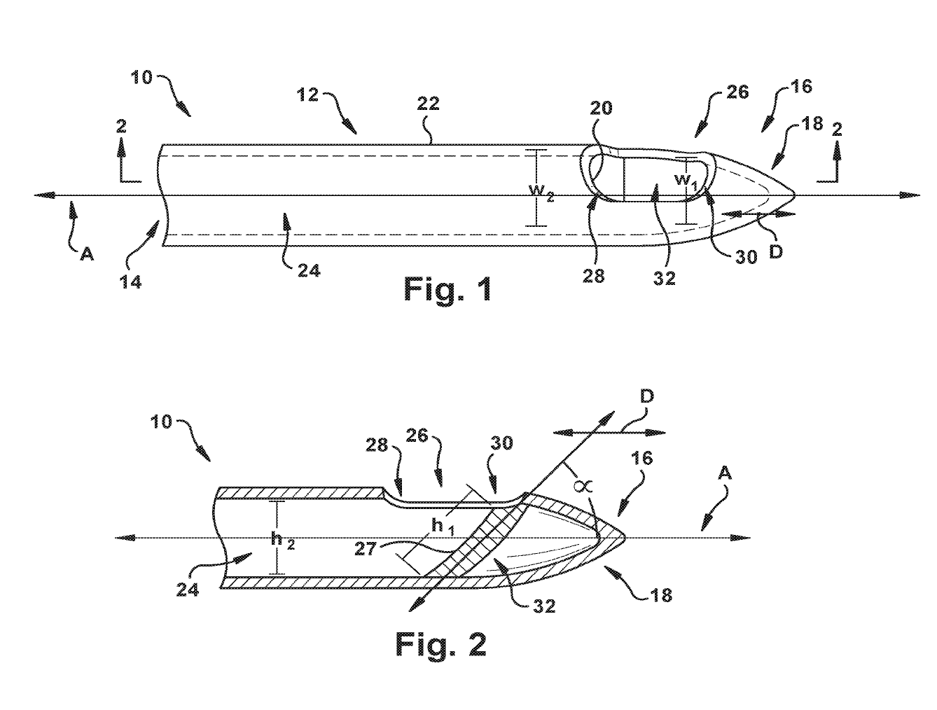

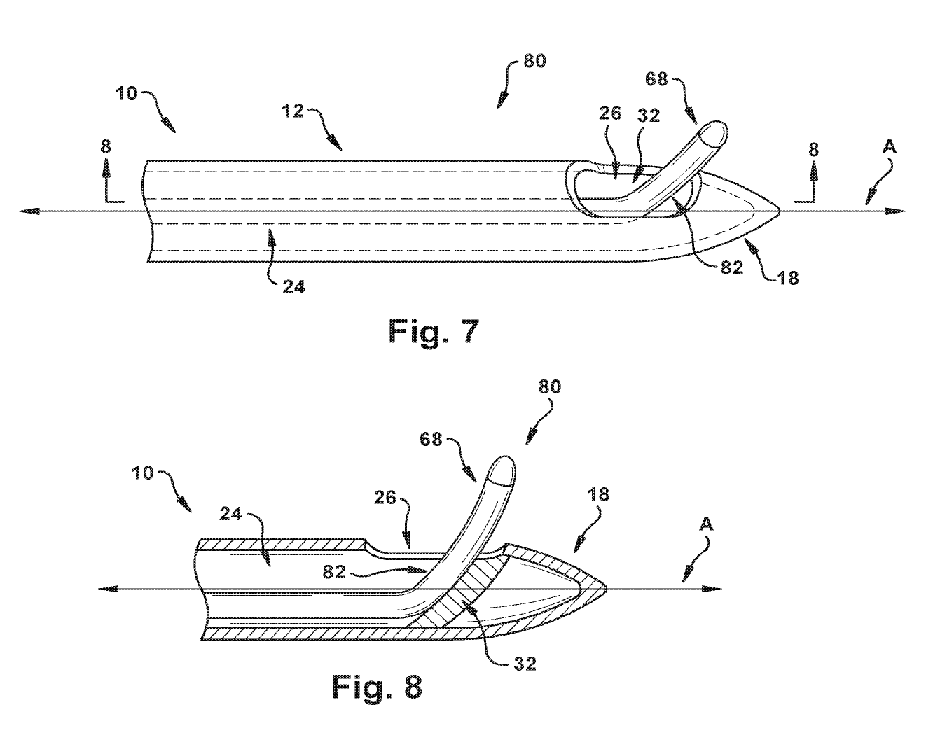

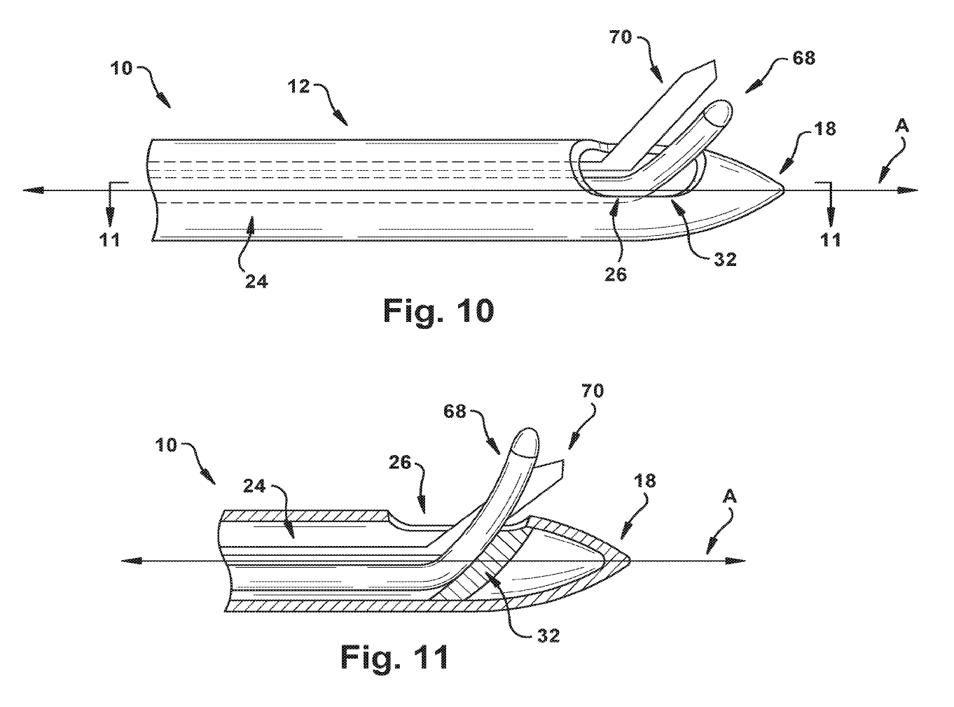

One aspect of the present disclosure can include a percutaneous needle 10 (FIGS. 1-2) for guiding a therapy delivery device into a target tissue. The percutaneous needle 10 can include a main needle body 12 having a proximal end portion 14 spaced apart from a distal end portion 16. In one example, the distal end portion 16 can include an illumination port (not shown) for conveying light out of the main needle body 12. In some instances, the distal end portion 16 can include a pointed tip 18 to facilitate penetration into a target tissue. The main needle body 12 can also include an inner surface 20 and an outer surface 22. The inner surface 20 can define an inner lumen 24 extending between the proximal and distal end portions 14 and 16 of the main needle body 12. In one example, the inner lumen 24 can extend along a longitudinal axis A of the main needle body 12. The inner lumen 24 can be sized and dimensioned to receive one or more surgical instruments 68 (FIG. 7-8) or 70 (FIGS. 10-11) (e.g., a camera, a therapy delivery device, etc.). The percutaneous needle 10 can have a tubular shape with a circular cross-sectional profile (or any other desired cross-sectional profile). The percutaneous needle 10 can be made from one or a combination of metals (e.g., aluminum, etc.) and/or non-metals (e.g., plastics, etc.). The percutaneous needle 10 can have a rigid or semi-rigid configuration.

In another aspect, the distal end portion 16 (FIGS. 1-2) of the main needle body 12 can include an opening 26 in communication with the inner lumen 24. The opening 26 can extend between the inner and outer surfaces 20 and 22 of the main needle body 12. The opening 26 can include a proximal portion 28 opposite a distal portion 30. As shown in FIGS. 1-2, the opening 26 can be axially spaced apart from a closed, pointed tip 18 of the main needle body 12. For example, the distal portion 30 of the opening 26 can be axially spaced apart from the tip 18 by a distance D. The opening 26 can be sized and dimensioned to permit passage of one or more surgical instruments 68 (FIG. 7-8) or 70 (FIGS. 10-11) therethrough. The opening 26 can a variety of shapes, including oval, circular, rectangular, square, etc.

In another aspect, the main needle body 12 can include a ramp 32 (FIGS. 1-2) disposed within the inner lumen 24. In some instances, the ramp 32 can extend between top and bottom portions of the inner lumen 24. For example, the ramp 32 can have a width w₁ and a height h₁ that spans a width w₂ and a height h₂ of the inner lumen 24. The ramp 32 can be positioned within the inner lumen 24 and have a surface 27 that extends at an angle a (e.g., between about 1° and about90°) relative to the longitudinal axis A of the main needle body 12. The ramp 32 can have a cross-sectional profile that is identical to, or substantially identical to, the cross-sectional profile of the main needle body 12. The ramp 32, and in particular the surface 27, can be axially spaced apart from the tip 18 and in communication with the opening 26. In one example, the ramp 32 (e.g., the surface 27) can be axially spaced apart from the tip 18 by a distance D. As shown in FIG. 2, the ramp 32 can be positioned adjacent the distal portion 30 of the opening 26 (e.g., so that a plane defined by the surface 27 intersects the opening 26). The ramp 32 can be configured to deflect one or more surgical instruments 68 (FIG. 7-8) or 70 (FIGS. 10-11) out of the opening 26 (e.g., by preventing the surgical instruments from extending past the ramp within the inner lumen 24) and away from the longitudinal axis A of the main needle body 12 when the surgical instrument(s) is advanced through the inner lumen 24 towards the tip 18.

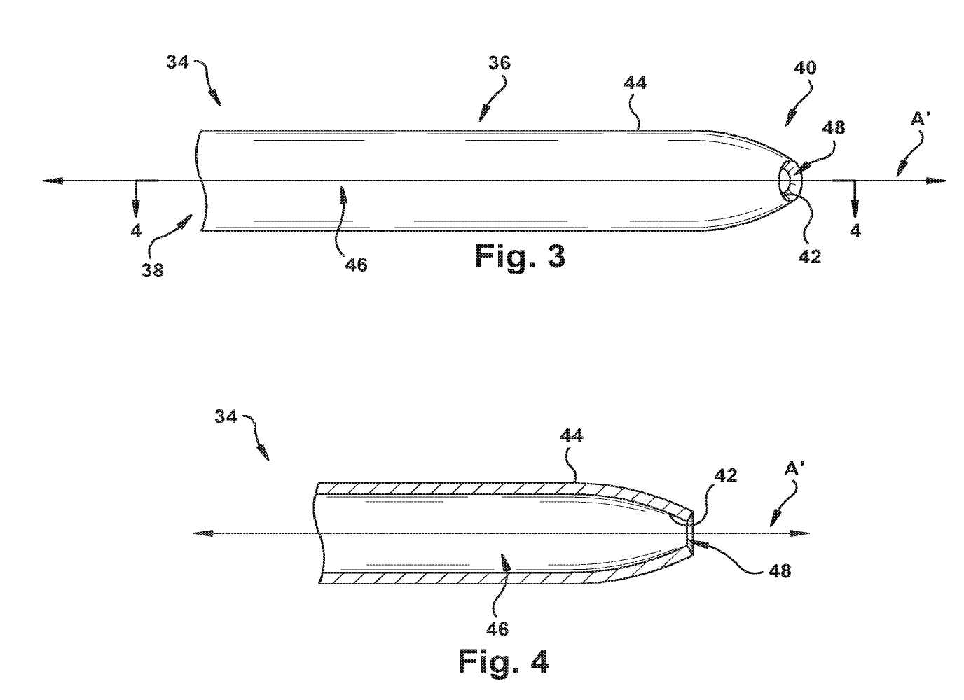

FIGS. 3-4 illustrate a percutaneous needle 34 constructed in accordance with another aspect of the present disclosure. Description of common elements and operation similar to those with respect to the percutaneous needle 10 will not be repeated for conciseness with respect to the percutaneous needle 34.

The percutaneous needle 34 can include a main needle body 36 having a proximal end portion 38 spaced apart from a distal end portion 40. The main needle body 36 can also include an inner surface 42 and an outer surface 44. The inner surface 42 of the main needle body 36 can define an inner lumen 46 extending between the proximal and distal end portions 38 and 40. The inner lumen 46 can extend along a longitudinal axis A′ of the main needle body 36. The inner lumen 46 can be sized and dimensioned to receive one or more surgical instruments. The percutaneous needle 34 can include an opening 48 located at the distal end portion 40 of the main needle body 36. As shown in FIGS. 3-4, the opening 48 can be substantially coaxial with the inner lumen 46. The opening 48 can be sized and dimensioned to permit passage of one or more surgical instruments 68 (FIG. 7-8) or 70 (FIGS. 10-11).

### Systems

Another aspect of the present disclosure can include an epidural needle system 80 (FIGS. 7-8). The epidural needle system 80 can comprise a percutaneous needle 10 and a camera 68. The percutaneous needle 10 can be identically or similarly constructed as the needle described above. For example, the percutaneous needle 10 can include a main body 12 defining an inner lumen 24 and an opening 26 located on a distal end portion 16 of the main body. The opening 26 can be in communication with the inner lumen 24. The needle 10 can also include a ramp 32 positioned within the inner lumen 24 adjacent the opening 26. The ramp 32 can be axially spaced apart from a tip 18 of the main body 12. The camera 68 can be slidably disposed within the inner lumen 24. The camera 68 can have a distal end portion 82 that overlies, and is in direct contact with, the ramp 32 (e.g., the surface 27 of the ramp) such that the ramp deflects the camera radially away from the distal end portion 16 of the needle 10.

### Methods

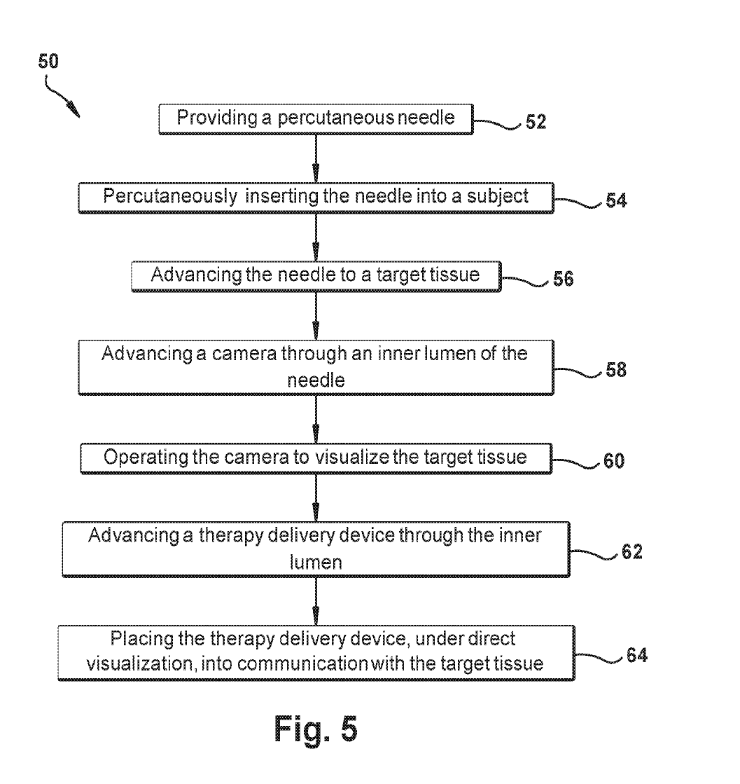

Another aspect of the present disclosure can include a method 50 (FIG. 5) for applying a therapy to a target tissue of a subject. The method 50 can generally include the steps of: providing a percutaneous needle (Step 52); percutaneously inserting the needle into a subject (Step 54); advancing the needle to a target tissue (Step 56); advancing a camera through an inner lumen of the needle (Step 58); operating the camera to visualize the target tissue (Step 60); advancing a therapy delivery device through the inner lumen (Step 62); and placing the therapy delivery device, under direct visualization, into communication with the target tissue (Step 64). It will be appreciated that the method 50 can be applied to a variety of medical or surgical procedures, such as epidural steroid injections, spinal cord stimulator placement procedures, and the like.

At Step 52, a percutaneous needle 10 can be provided. For the purpose of illustration only, the method 50 will be described below using the percutaneous needle 10 shown in FIG. 1 and described above. It will be appreciated that the particular dimensions of the percutaneous needle 10 will depend upon the particular epidural medical or surgical procedure.

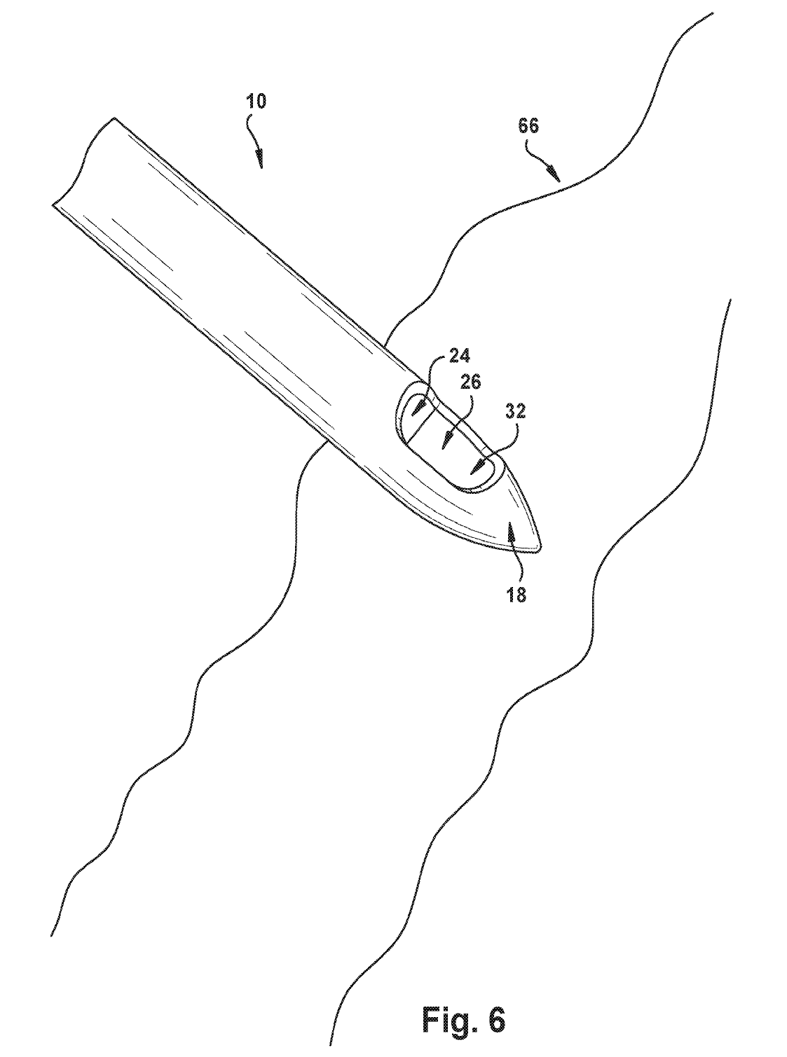

At Step 54, the percutaneous needle 10 can be inserted into a subject. As shown in FIG. 6, the distal end portion 16 of the main needle body 12 can be placed into contact with the skin of the subject and then slightly advanced so that the pointed tip 18 penetrates the skin.

At Step 56, the needle 10 can be advanced to a target tissue 66 (e.g., the epidural space) of the subject. The needle 10 can be urged through the subject until the distal end portion 16 of the needle 10 is positioned adjacent the target tissue 66 (FIG. 6).

At Step 58, a camera 68 can be advanced through the inner lumen 24 of the needle 10. The camera 68 can be any suitable camera, such as a 0.99 mm camera (available from Medigus Ltd., Omer, Israel), a miniature CMOS image sensor module (available from Fujikura Ltd., Duncan, S.C.), or a naneye device (available from Awaiba Lda, Madeira, Portugal). As shown in FIGS. 7-8, the ramp 32 can deflect the camera 68 out of the opening 26 upon passage of the camera 68 through the inner lumen 24.

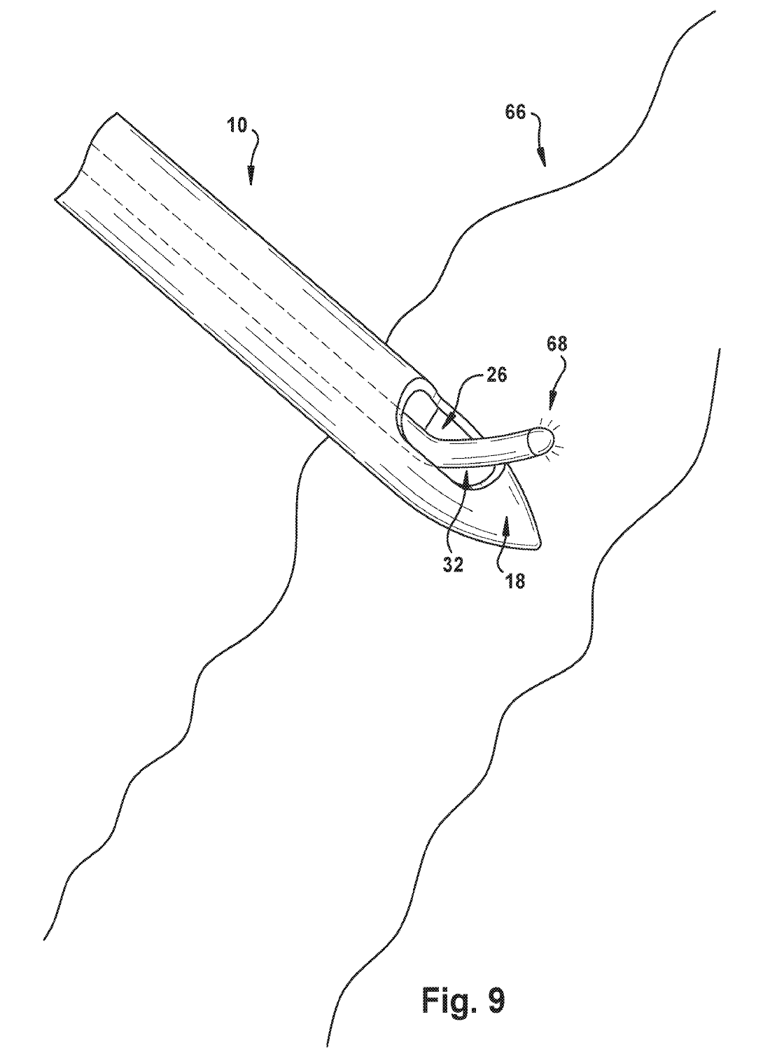

At Step 60, the camera 68 can then be operated to visualize the target tissue 66 (FIG. 9) (e.g., the yellow ligament and dura layer).

At Step 62, a therapy delivery device 70 can be advanced through the inner lumen 24. The therapy delivery device 70 can include an endoscope, a hypodermic needle containing a therapeutic agent, a catheter, a neurostimulation lead, etc. As shown in FIGS. 10-11, the ramp 32 can deflect the therapy delivery device 70 out of the opening 26 upon passage of the therapy delivery device 70 through the inner lumen 24. The camera 68 and the therapy delivery device 70 can then be simultaneously or sequentially advanced through the inner lumen 24 and deflected out of the opening 26 by the ramp 32. Advantageously, advancement of the camera 68 and the therapy delivery device 70 reduces the risk of dura layer puncture by visualizing the target tissue 66 prior to placing the therapy delivery device into communication with the target tissue.

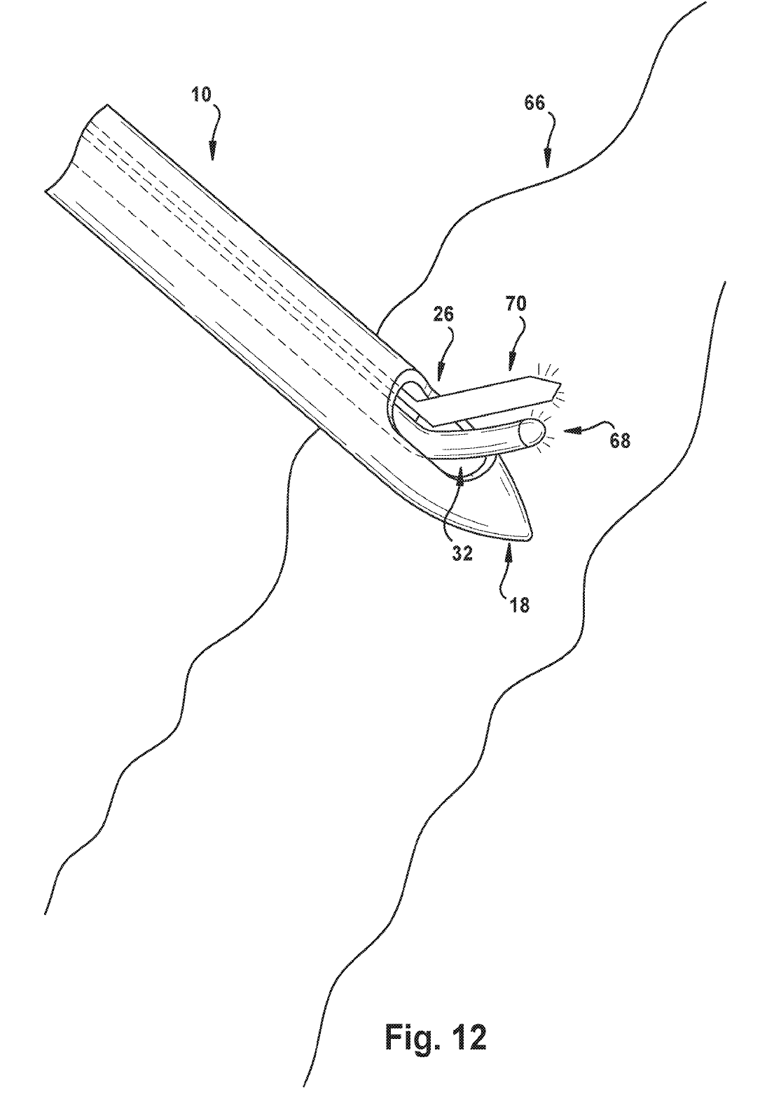

At Step 64, the therapy delivery device 70 can then be placed, under direct visualization, into communication with the target tissue 66 (FIG. 12). In one example, the therapy delivery device 70 can be a hypodermic needle for delivering a therapeutic agent (e.g., a steroid) to the target tissue 66. In another example, the therapy delivery device can be a neurostimulation lead for delivering stimulation to the target tissue 66. Once the therapy is delivered to the target tissue 66, the percutaneous needle 10, the camera 68, and the therapy delivery device 70 can be removed from the subject to complete the medical or surgical procedure.

From the above description of the present disclosure, those skilled in the art will perceive improvements, changes, and modifications. Such improvements, changes, and/or modifications are within the skill of the art and are intended to be covered by the appended claims.

1. A percutaneous needle for guiding a therapy delivery device to a target tissue in a subject, the needle comprising:
a main body defining an inner lumen;
an opening located on a distal end portion of the main body, the opening being in communication with the inner lumen; and
a ramp positioned within the inner lumen and adjacent the opening, the ramp being axially spaced apart from a distal end of the main body.

2. The needle of claim 1, further comprising a camera slidably disposed within the inner lumen and having a distal end portion that overlies, and is in direct contact with, the ramp such that the ramp deflects the camera radially away from the distal end portion of the needle.

3. The needle of claim 2, further comprising a therapy delivery device disposed within the inner lumen.

4. The needle of claim 1, wherein the inner lumen is sized and dimensioned to house a camera and a therapy delivery device therein.

5. The needle of claim 1, wherein the opening is axially spaced apart from a pointed tip of the needle.

6. The needle of claim 1, wherein the ramp extends between top and bottom portions of the inner lumen.

7. The needle of claim 6, wherein the ramp has a surface that extends at an angle of between about 1° and about 90° relative to a longitudinal axis of the main body.

8. The needle of claim 6, wherein the ramp has a cross-sectional profile that is identical to a cross-sectional profile of the inner lumen.

9. An epidural needle system comprising:
a percutaneous needle including a main body defining an inner lumen, an opening located on a distal end portion of the main body, the opening being in communication with the inner lumen, and a ramp positioned within the inner lumen and adjacent the opening, the ramp being axially spaced apart from a tip of the main body; and
a camera slidably disposed within the inner lumen and having a distal end portion that overlies, and is in direct contact with, the ramp such that the ramp deflects the camera radially away from the distal end portion of the needle.

10. The epidural needle system of claim 9, further comprising a therapy delivery device disposed within the inner lumen.

11. The epidural needle system of claim 9, wherein the opening is axially spaced apart from a pointed tip of the needle.

12. The epidural needle system of claim 9, wherein the ramp extends between top and bottom portions of the inner lumen.

13. The epidural needle system of claim 12, wherein the ramp has a surface that extends at an angle of between about 1° and about 90° relative to a longitudinal axis of the main body.

14. The epidural needle system of claim 12, wherein the ramp has a cross-sectional profile that is identical to a cross-sectional profile of the inner lumen.

15. A method for guiding a therapy delivery device to a target tissue in a subject, the method comprising the steps of:
providing a needle comprising:
a main body defining an inner lumen;
an opening located on a distal end portion of the main body, the opening being in communication with the inner lumen; and
a ramp positioned within the inner lumen and adjacent the opening, the ramp being axially spaced apart from a distal end of the main body;
percutaneously inserting the needle into the subject;
advancing the needle to the target tissue;
advancing a camera through the inner lumen;
operating the camera to visualize the target tissue;
advancing a therapy delivery device through the inner lumen; and
placing the therapy delivery device, under direct visualization, into communication with the target tissue.

16. The method of claim 15, wherein the step of providing the needle further comprises the step of:
axially spacing the ramp from a distal end of the main body.

17. The method of claim 15, wherein the step of advancing a camera through the inner lumen further comprises the step of:
advancing the camera over the ramp such that the ramp deflects the camera away from the distal end portion of the needle towards the target tissue.

18. The method of claim 15, wherein the step of advancing a therapy delivery device through the inner lumen further comprises the step of:
advancing the therapy delivery device over the ramp such that the ramp deflects the therapy delivery device away from the distal end portion of the needle towards the target tissue.

19. The method of claim 18, wherein the camera is removed from the inner lumen prior to advancing the therapy delivery device through the inner lumen.

20. The method of claim 15, wherein the inner lumen is sized and dimensioned to separately or collectively accommodate each of the camera and the therapy delivery device.

21. The method of claim 15, further comprising the step of applying a therapeutic to the target tissue via the therapy delivery device.
